SEQUENCE LISTING (4 sequences)

SEQ ID NO: 1 - DNA, 18 bp, Artificial
  Probe sequence for Hybridization test
actggccgtcgttttaca

SEQ ID NO: 2 - DNA, 18 bp, Artificial
  Probe sequence for Hybridization test
actggccgttgttttaca

SEQ ID NO: 3 - DNA, 18 bp, Artificial
  Probe sequence for Hybridization test
actggccgcttttttaca

SEQ ID NO: 4 - DNA, 18 bp, Artificial
  Probe sequence for Hybridization test
actggcatcttgtttaca

(19) United States
(12) Patent Application Publication
(10) Pub. No.: US 2007/0111250 A1
(43) Pub. Date: May 17, 2007

(54) Probe carrier, method of producing the probe carrier, method of evaluating the probe carrier and method of detecting a target nucleic acid using the same

(75) Inventor: Kazuhiro Takada, Kanagawa (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/648,602

(22) Filed: Jan. 3, 2007

Related U.S. Application Data
(62) Division of application No. 10/725,396, filed on Dec. 3, 2003.
Application No. 10/725,396 is a continuation of application No. PCT/JP03/08196, filed on Jun. 27, 2003.

(30) Foreign Application Priority Data
Jun. 28, 2002 (JP) 2002-191224

(51) Int. Cl. C12Q 1/68; C12M 3/00

(52) U.S. Cl. 435/6; 435/287.2; 977/924

(57) ABSTRACT

To provide probe carrier having a structure that a single-stranded DNA probe can be immobilized to a carrier effectively and forms of probe dots or spots immobilized to the carrier can be observed accurately by a scanning probe microscope or its derivative technology. A gold-containing film is formed in an area of the carrier where the single-stranded DNA probe is immobilized and the single-stranded DNA probe is immobilized to this film through a sulfur atom.

This application is a continuation of International Application No. PCT/JP03/08196, filed Jun. 27, 2003, which claims the benefit of Japanese Patent Application No. 2002-191224, filed Jun. 28, 2002.

### BACKGROUND OF THE INVENTION

1. Field of the Invention

The present invention relates to a probe carrier having a single-stranded DNA probe immobilized to a gold-containing film, a method of producing the same, a method of evaluating the same, and a method of detecting a target nucleic acid using the same.

2. Related Background Art

As one of techniques capable of carrying out a determination of a base sequence of a nucleic acid, a detection of a target nucleic acid contained in a sample and an identification of various bacteria quickly and accurately, there is proposed, for example, use of a substance that can be specifically bond to a target nucleic acid, i.e., a so-called “probe array” having a large number of probes arranged on a solid phase. As methods of producing this probe array, there are known the following general ones:

(1) one in which a nucleic acid probe is synthesized on a solid phase; and

(2) one in which the nucleic acid probe synthesized in advance is supplied to the top of a solid phase.

The prior art which discloses details of the above method (1) includes, for example, U.S. Pat. No. 5,405,783.

The prior art which discloses the above method (2) includes, for example, U.S. Pat. No. 5,601,980 and “Science”, Vol. 270, pp. 467 (1995), all of which disclose a method of arranging cDNA's in an array using micropipetting.

Since the nucleic acid probe is directly synthesized on the solid phase in the above method (1), the nucleic acid probe does not need to be synthesized in advance. However, it is difficult to purify the probe nucleic acid synthesized on the solid phase. The accuracy of the determination of the base sequence of a nucleic acid or the detection of a target nucleic acid contained in a sample by using a probe array greatly depends on the accuracy of the base sequence of a nucleic acid probe. Therefore, the above method (1) is desirably further improved to increase the accuracy of the nucleic acid probe as a method of producing a higher-quality probe array.

Although the above method (2) requires a step of synthesizing a nucleic acid probe prior to an immobilization of the nucleic acid probe to the solid phase, the nucleic acid probe can be purified before it is bound to the solid phase. For this reason, in a current stage, the above method (2) is more preferred than the above method (1) as the method of producing the higher-quality probe array.

However, in the above method (2), it is necessary to spot the nucleic acid probe to the solid phase at a high density. For example, when the base sequence of a nucleic acid is determined by using a probe array, it is preferred to arrange as many nucleic acid probes as possible on the solid phase. To detect variation of a gene efficiently, nucleic acid probes having sequences corresponding to variations are preferably arranged on the solid phase. Further, for the detection of a target nucleic acid contained in a sample and the detection of the variation and loss of the gene, an amount of the sample from a subject, specifically blood or the like is preferably as small as possible. Therefore, it is preferred that as much information on base sequences as possible can be obtained from a small amount of a sample.

It is desired that a surface of a substrate to which the nucleic acid probes are immobilized by the above method (2) have a function of firmly binding the probes thereto and smoothness. Materials which have been conventionally and frequently used include glass, plastics (such as polypropylene, polystyrene, polycarbonate and mixtures thereof), and metals (such as gold and platinum). Since chemical bonding is generally used to directly immobilize the probes to the surface of the substrate, functional groups at binding sites between the probes and the surface of the substrate are selected from combinations of functional groups having high reactivity. Therefore, a binding layer for binding the probes thereto must be formed on the surface of the substrate. In most cases, a binding layer having a thickness ranging from a monomolecular size to about 1 mm is formed as a single layer or a multi-layer to firmly immobilize the probes to the substrate.

Since the probes themselves formed on the substrate have a nanometer-order size structure, to observe or evaluate them, scanning probe microscope has often been used. The term “scanning probe microscope” as described herein is a general term for scanning microscope which enables an observation of a sample to an atomic level by bringing a fine needle to the surface of the sample and means scanning tunneling microscope (STM) and atomic force microscope (AFM), inclusive of an observation technique using the microscope which is derived from those technologies, or the like. As for the observation of DNA by the scanning probe microscope, there have already been a large number of reports. However, a metal substrate should be treated in ultra high vacuum to form DNA on the substrate, that is, a sample must be produced in a state suitable for observation.

### SUMMARY OF THE INVENTION

In the above prior art method of forming probes, however, the step of forming the binding layer is required to form binding layer for firmly binding the probes to the substrate on the substrate, in addition to the treatment of the substrate. Therefore, the method takes a lot of time and labor to form the probes. Since surface unevennesses of the binding layer are reflected when the surface of each of the thus formed probes is observed through the scanning probe microscope and the like, this method is not suitable for the observation of a shape of a nanometer-order size probe itself.

It is therefore an object of the present invention which has been made to solve the above problems to provide a method of forming a carrier which is easily formed and has a smooth surface and efficiently and accurately immobilizing an extremely small amount of a single-stranded DNA probe to the carrier without damaging the probe and without forming a binding layer and the like on the carrier.

It is another object of the present invention to provide a probe carrier such as a probe array capable of accurately detecting more information on DNA even from a small amount of a sample.

It is still another object of the present invention to provide a probe carrier having such a structure that enables a shape or the like of a probe array on a carrier to be observed or inspected accurately using scanning probe microscope and a technique derived therefrom.

The present invention has been completed by conducting intensive studies to solve the above problems and is constituted as will be described below.

That is, according to the present invention, there is provided a probe carrier, characterized in that a single-stranded DNA probe is immobilized to a carrier having a thin film containing gold formed thereon through a sulfur atom.

According to the present invention, there is provided a method of producing a probe carrier having a single-stranded DNA probe immobilized to a carrier having a thin film containing gold formed thereon through a sulfur atom, characterized by including the steps of:

forming the thin film containing gold on the carrier; and

immobilizing the single-stranded DNA probe to the thin film through the sulfur atom.

According to the present invention, there is provided method of evaluating a probe carrier, characterized by including observing and inspecting a form of the probe carrier produced by the above-mentioned method through a scanning probe microscope.

According to the present invention, there is provided method of detecting a target nucleic acid using a probe carrier having a single-stranded DNA probe for the detection of the target nucleic acid, characterized in that the probe carrier has the above-mentioned constitution.

According to the present invention, since a single-stranded DNA probe having a thiol group as a functional group is immobilized to a gold-containing film capable of forming a smooth surface, a strong bond is formed by linkage between gold and the thiol group through a sulfur atom, thereby making it possible to provide a probe carrier having a single-stranded DNA probe firmly bound to a carrier. Since the gold-containing film used in the present invention has extremely high flatness and is so stable that it is hardly oxidized in the air, probes can be directly evaluated by a microscope having atomic resolution such as a scanning probe microscope.

According to the present invention, when the above gold-containing film is used as the electrodes which can be applied with voltage, a method of detecting a hybrid substance (double-stranded nucleic acid) by electrochemical measurement using the electrodes, a molecular device produced by interposing a DNA between the electrodes, and the like may be obtained.

### BRIEF DESCRIPTION OF THE DRAWINGS

FIG. 1 is a schematic diagram of a gold crystal thin film forming apparatus.

FIG. 2 is a schematic diagram of an apparatus for carrying out production of a patterned substrate.

FIG. 3A is a schematic plan view of a gold patterned substrate, and FIG. 3B is a sectional view taken along the line 3B-3B of FIG. 3A after DNA is spotted.

FIG. 4 is a schematic diagram of a patterning apparatus using an electron beam, which will be explained in Example 3.

### DETAILED DESCRIPTION OF THE PREFERRED EMBODIMENTS

In the present invention, a probe immobilized to a carrier can be specifically bound to a specific target substance, and when the probe has a complementary sequence to a base sequence of a target nucleic acid a hybrid substance thereof can be formed.

The carrier can be selected from carriers having various forms, made from various materials and capable of forming a gold-containing film. For example, a glass and the like substrate can be advantageously used.

The nucleic acid constituting the probe is a single-stranded DNA, which may be synthesized, or a portion having a desired probe function taken out from a genome DNA or cDNA.

The immobilization of the single-stranded DNA probe to the carrier is carried out through a reaction between a gold-containing film and a thiol group as will be described hereinafter. It is preferred to take into consideration a position of a binding site thereof to prevent the from affecting hybridization.

In the present invention, what has a probe immobilized area where a probe is immobilized in a dot or spot form on a carrier is called “probe carrier” and what has a plurality of probe immobilized areas arranged in a matrix form at predetermined positions on a carrier independently of each other is called a “probe array”. This probe carrier includes what is generally called a “micro-array” or a “nucleic acid chip” such as a DNA chip.

In the present invention, each probe-immobilized area is as large as 5 to 500 μm², preferably 10 to 200 μm².

The gold-containing film is preferably a (111)-oriented single crystal thin film, more preferably a gold single crystal thin film having a surface unevenness of 0.5 nm or less per μm² and a thickness of 5 μm or less. A sulfur atom interposed between the carrier and the single-stranded DNA probe is preferably introduced as the functional group of the single-stranded DNA probe. To obtain the gold-containing film, a method in which a carrier is immersed in a gold complex solution to form a gold single crystal thin film on the carrier can be advantageously employed.

A preferred embodiment of the present invention will be described hereinbelow with reference to the accompanying drawings.

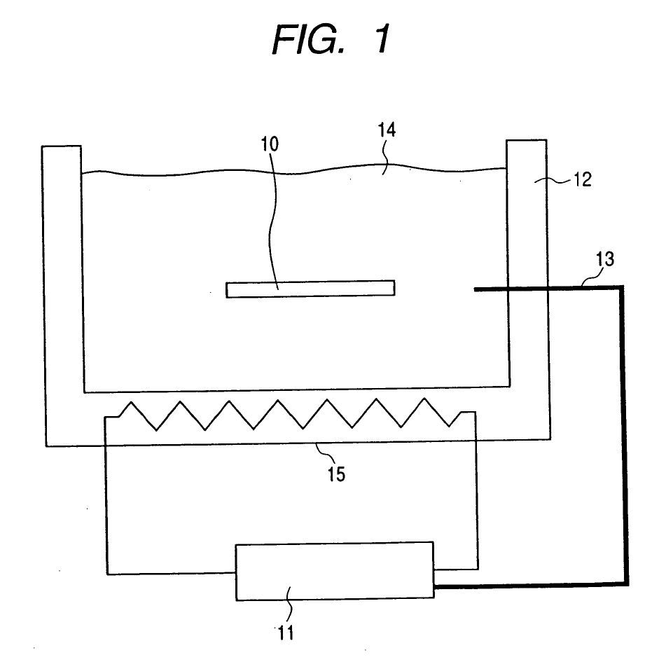

FIG. 1 is a schematic diagram of a gold crystal thin film forming apparatus for selectively depositing gold single crystals on a substrate as a carrier. In FIG. 1, reference numeral 12 denotes a solution tank; 14, a solution; 13, a temperature measuring device such as a thermocouple for measuring the temperature of the solution; 15, a heater for heating the solution 14; and 11, a power source having a mechanism for maintaining the temperature of the solution at a constant value by controlling voltage to be applied to the heater is based on a temperature signal obtained from the thermocouple 13.

The method of forming the DNA chip of the present invention using the above apparatus will be described hereinbelow. A description is first given of the step of forming a substrate.

A gold thin film is first formed on the substrate. The substrate may be made from various kinds of glass, metal or silicon. Distilled water is first poured into the solution tank 12, potassium iodide and iodine are added to prepare an iodine aqueous solution, gold is added, stirred and dissolved in the above solution to prepare a gold complex solution containing [AuI₄]⁻ as the solution 14. At this point, it is considered that I₃⁻ and K⁺ are existent in the solution in addition to the gold complex [AuI₄]⁻.

The iodine aqueous solution can be also prepared by dissolving an iodide compound other than potassium iodide, such as ammonium iodide. An iodine alcohol solution prepared by using an alcohol as a solvent and an iodine alcohol-aqueous solution prepared by using a mixture of alcohol and water as a solvent may also be used in the present invention. The content of iodine or iodide compound in the solution affects the amount of gold which can be dissolved.

Then, the surface of the above substrate 10 is contacted to the solution, the solution 14 is heated to reach a desired temperature of 30 to 100° C. by the heater 15, and the temperature of the solution 14 is controlled by the power source 11 so that it becomes constant to promote a volatilization of the iodine component.

In the solution 14 system, it is considered that decomposition proceeds due to a dissociation of the iodine component from [AuI₄]⁻ for maintaining equilibrium in the solution system caused by the volatilization of the iodine component existent in the form of I₃⁻, or decomposition proceeds due to the direct volatilization of the iodine component contained in the complex existent in the form of [AuI₄]⁻. As a result, gold is supersaturated.

Gold that becomes super saturated in the solution 14 is deposited on the surface of the substrate as random nuclei and the nuclei grow in a self-aligning manner to form a single crystal film.

When an X-ray diffraction measurement of the formed gold single crystal film was carried out, it was found that the single crystals had no defect and were (111)-oriented.

A single-stranded DNA probe is then formed on the thus formed substrate. A DNA probe having a thiol group bonded thereto is first synthesized. For example, 5′-Thiol-Modifier C6 (produced by Glen Research Co., Ltd.) can be used for synthesis as a 5′ terminal reagent when DNA is automatically synthesized by a DNA automatic synthesizer. After an ordinary deprotection reaction, it can be obtained by purification by high pressure liquid chromatograph. The spotting of the DNA probe to the gold substrate is carried out by discharging it by using an ink jet technique.

A liquid composition to be spotted is preferably contained in a suitable concentration in consideration of an ink jet discharge characteristics of the liquid and the stability of the DNA probe in the liquid and at the time of bubble jet discharge.

The composition of the liquid to be discharged from a bubble jet head is not particularly limited if, when the liquid is mixed with a DNA probe as described above and discharged from the bubble jet head, it does not substantially affect the DNA probe and can be discharged properly to a solid phase by using the bubble jet head in a satisfactory condition. The liquid is preferably a liquid containing glycerin, urea, ethylene glycol, isopropyl alcohol and acetylene alcohol represented by the following formula (I): 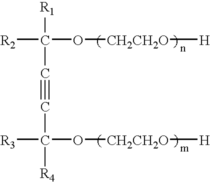
(wherein R₁, R₂, R₃ and R₄ are each an alkyl group, specifically, linear or branched alkyl group having 1 to 4 carbon atoms, for example, and m and n are each an integer, with the proviso that m=0 and n=0, or 1≦m+n≦30, or m or n is 0 when m+n=1.)

More specifically, a liquid containing 5 to 10% by weight of urea, 5 to 10% by weight of glycerin, 5 to 10% by weight of ethylene glycol and 0.02 to 5% by weight, more preferably 0.5 to 1% by weight of acetylene alcohol represented by the above formula (I) is advantageously used.

When this liquid is used and a mixture of this liquid and a nucleic acid probe is discharged from the bubble jet head and adhered to a solid phase, a thiol group contained in the liquid is selectively bound to a gold thin film. A reaction between gold and thiol is considered to occur based on the following formula.
Carrier-Au+DNA-SH→Au—S-DNA+½H₂

Due to the above selective reaction, the single-stranded DNA probe is selectively bound only to gold formed on the substrate to form a probe carrier.

The thus formed probe array may be constituted to have a plurality of spots containing the same DNA probe or to have a plurality of spots containing different DNA probes according to its application purpose. The probe array having DNA probes arranged at a high density produced by this method is used for the detection of a target single-stranded DNA and the identification of a base sequence thereafter. For example, when it is used for the detection of a target single-stranded DNA having a known base sequence, which may be contained in the sample, a single-stranded DNA having a complementary base sequence to the base sequence of the target single-stranded DNA is used as a probe, a probe array having a plurality of spots containing the probe arranged on a solid phase is prepared, a sample is supplied to each spot of the probe array to be placed under conditions that the target single-stranded DNA and the DNA probe can be hybridized, and the existence of a hybrid at each spot is detected by a known method such as fluorescent detection. As a result, the existence of a target substance contained in the sample can be detected. When it is used for the identification of the base sequence of a target single-stranded DNA contained in a sample, a plurality of base sequences considered the same as the base sequence of the target single-stranded DNA are set and single-stranded DNA's having complementary base sequences to the above base sequences are spotted as probes to a solid phase. Then, the sample is supplied to each spot to be placed under conditions that the target single-stranded DNA and the DNA probes can be hybridized, and the existence of a hybrid at each spot is detected by a known method such as fluorescent detection. As a result, the base sequence of the target single-stranded DNA can be identified.

The method of forming the single-stranded DNA probe on the substrate with the above procedure has been described. A feature of this substrate is that the single-stranded DNA probe bound surface of gold has extremely high smoothness. In addition, since gold is used, the surface of the substrate is hardly oxidized and retains its smooth surface stably even when it is left in the air. Making use of this feature, the form of the single-stranded DNA probe can be directly observed through a microscope having atomic resolution such as a scanning probe microscope, for example. A DNA chip having a substrate made from an insulating material can be observed only through an atomic force microscope whereas this DNA chip can be observed through STM for monitoring a tunnel current because the substrate of the DNA chip has conductivity.

The probe carrier according to the present invention enables a hybridization reaction between a DNA probe bound to gold and a target single-stranded DNA to be detected electrochemically by using the formed gold-containing film as electrodes.

A gene detection method using the film as electrodes will be described in detail hereinbelow.

It is also possible to provide a molecular device by using the formed gold-containing film as electrodes and spotting DNA having a thiol group at both terminals between adjacent electrodes.

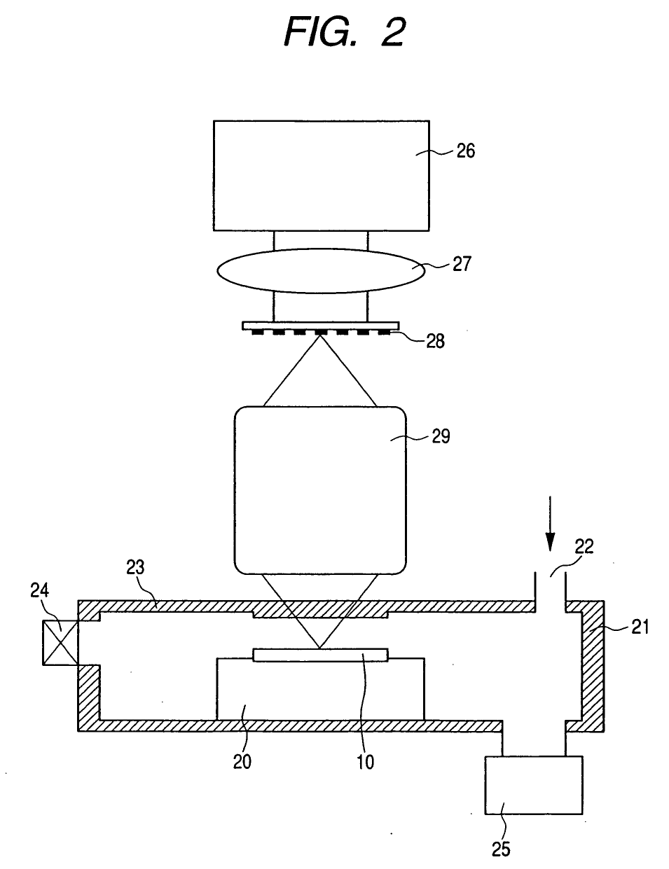

A description is subsequently given of a method of forming a DNA chip by patterning gold. FIG. 2 is a schematic diagram of an apparatus for carrying out the production of a patterned substrate. A method of forming and using a latent image including an area where the gold thin film can be formed by applying an electron beam and ion and an area where a gold thin film cannot be formed is advantageously used for the pattering of the gold-containing film.

In FIG. 2, reference numeral 20 denotes a sample holder; 21, a latent image chamber which can be made airtight under vacuum; 22, a gas introduction port for introducing a reactive gas required for the formation of a latent image layer on a substrate; 24, a gate valve which enables the airtightness under vacuum for taking a substrate 10 out from and into the latent image chamber 21; 25, a vacuum evacuator which can evacuate the inside of the latent image chamber 21 and can control its evacuation rate; 26, a light source as a source for generating an energy beam (in this case, KrF excimer laser); 28, an illumination optical system for applying an excimer laser beam through a mask 27 having a desired pattern; 29, a projection optical system for forming an image of the mask on the surface of the substrate 10; and 23, a light incident window which enables the made airtightness under vacuum, transmits the excimer laser beam and is made from molten quartz, in this case.

In order to produce a gold patterned substrate, the gate valve 24 is first opened, the substrate 10 such as a silicon substrate is mounted on the sample holder 20, and the gate valve 24 is closed to evacuate the latent image chamber 21 by the vacuum evacuator 25 until an inside pressure of the latent image chamber 21 becomes 10⁻⁷ Torr or less. A latent image forming gas such as O₂ is introduced into the latent image chamber 21 from the gas introduction port 22, and the evacuation rate of the vacuum evacuator 25 is controlled so that the inside pressure of the latent image chamber 21 becomes a predetermined value in the range of 0.1 to 760 Torr. Thereafter, a laser beam having a wavelength of 248 nm oscillated from the KrF excimer laser 26 is uniformly applied to the mask 27 having a desired pattern by the illumination optical system 28, and a pattern image of the mask 27 is formed on the substrate 10 by the projection optical system 29 through the light incident window 23. On the surface of the substrate on which the mask image is formed, a photochemical reaction between the latent image forming gas and the Si substrate occurs only in exposed portions to form a latent image layer. Since O₂ is used as the latent image forming gas, the latent image layer is composed of silicon oxide. After the latent image layer having a desired thickness (2 to 10 nm) is formed, the supply of the gas is stopped, and the latent image chamber 21 is evacuated until the inside pressure of the latent image chamber 21 becomes 10⁻⁷ Torr or less. The gate valve 24 is opened to take out the substrate 20. The KrF excimer laser is used as a light source but the light source is not particularly limited if it has a wavelength for causing a photochemical reaction on the surface of the sample. Lamp light sources such as xenon lamp and high-pressure mercury lamp, and light sources such as ArF excimer laser, XeCl excimer laser, and Ar laser may also be used. Molten quartz is used as the material of the light incident window 23 to transmit a laser beam having a wavelength of 248 nm without absorbing it. CaF₂, MgF₂, LiF₂ and sapphire glass may also be used. In other words, any material is acceptable with no particular limitation if it transmits light emitted from a light source and withstands a pressure difference between the inside and outside of the latent image chamber.

Gold is then formed on the substrate produced in the above step. The above-described method may be used directly to form gold on the substrate. Using the thin film forming apparatus shown in FIG. 1, the substrate 10 is immersed in the solution tank 12. Gold which becomes supersaturated in the solution 14 is deposited only on the surface of the substrate having a high nucleus formation density where the latent image layer is not formed as random nuclei which grow in a self-aligining manner to form a single crystal film. Meanwhile, the surface of the latent image layer is made from SiO₂, and gold single crystals are not formed on the latent image layer because the SiO₂ surface has a low nucleus formation density.

When the exposed portion of Si is 40 μm or more in diameter, growth starts from the plurality of nuclei, and crystals collide with one another to form a grain boundary and cover a surface made from a material having a high nucleus formation density, whereby a gold crystal thin film made from single crystals having an average grain size of about 80 to 100 μm can be selectively formed.

After the patterned gold thin film is thus formed on the substrate, the substrate is then mounted on a stage or the like which can be moved two-dimensionally, and a DNA probe having a thiol group bonded thereto is discharged by an ink jet technique so that a probe nucleic acid is formed based on the gold pattern, thereby making it possible to produce a DNA chip.

Further, since the above gold thin film is used as electrodes, wirings to be electrically connected to the thin film may be further formed. The wirings may be formed by using a known technique, for example, a silver paste wiring may be connected to the gold thin film pattern.

As for the DNA chip having electrodes according to the present invention, a hybridization reaction can be promoted by applying a potential to the surfaces of the electrodes before and/or during the hybridization reaction.

A voltage to be applied is preferably a positive potential or AC potential and applied continuously or intermittently like pulses. The potential to be applied may be constant or variable, but preferably −0.2 to +2.0 V, more preferably 0 to +1.0 V positive potential.

The present invention also provides a method of detecting a gene using the above electrodes.

A description is subsequently given of the method of detecting the gene using the gold-containing film as the electrodes.

A double-stranded nucleic acid is formed on a carrier through a hybridization reaction between a nucleic acid probe immobilized to the carrier and a target nucleic acid. To detect this double-stranded nucleic acid, a method of causing an intercalating agent or a biopolymer for identifying a double-stranded nucleic acid to act or a method of introducing a label which is electrochemically detectable into a target nucleic acid may be used.

Examples of the substance called the “intercalating agent” include a substance which has a plate-like intercalating group such as a phenyl group in the molecule, like a tris(bipyridyl)cobalt complex and in which this intercalating group can interpose between base pairs of a double-stranded nucleic acid can be given.

Among the intercalating agents, there is a substance which responds to an electrode. An intercalating agent bound to a double-stranded nucleic acid can be detected by the measurement of an optical change or electrochemical change.

To detect an electrochemical change using electrodes, a metal complex which contains a substance causing an electrically reversible oxidation/reduction reaction as a central metal, that is, metallo-intercalating agent may be used as the intercalating agent besides the above substance in which the intercalating agent itself is reversible in an oxidation/reduction reaction. In such an intercalating agent, it is desired that the oxidation/reduction potential of the center metal of the complex or the intercalating agent itself be not equal to or higher than the oxidation/reduction potential of a nucleic acid or not the same as the oxidation/reduction potential of a nucleic acid.

By using the intercalating agent which causes an electrochemically reversible oxidation/reduction reaction, an oxidation/reduction current can be measured repeatedly. Therefore, the scanning of a potential is repeated several times to several hundreds of times to integrate the obtained signal values, thereby making it possible to amplify the signal. As a result, higher sensitive detection is enabled.

Further, an intercalating agent for causing electrochemical light emission such as an acridinium derivative may be used. An optical signal produced by electrochemical light emission may be directly detected from a solution by a photon counter, for example.

An electrode reaction or a change in optical signal can be extremely simply detected without removing an unreacted probe or unreacted intercalating agent because it occurs only on the electrode formed surface.

In the present invention, a reaction between the nucleic acid probe and the single-stranded sample nucleic acid is generally carried out in a solution. A hybridization reaction between the nucleic acid probe and the sample nucleic acid may be carried out in the presence of the above intercalating agent, or the intercalating agent may be added after the end of the above reaction.

Separate from this, among biopolymers including a DNA binding protein like an anti-DNA antibody, there are substances which recognize a double-stranded nucleic acid and are specifically bound to the double-stranded nucleic acid. Therefore, a double-stranded nucleic acid can be detected by binding a labeling substance such as an enzyme, fluorescent substance or luminescent substance to such a biopolymer or a substance which recognizes this biopolymer, measuring an electrochemical or optical change caused by this labeling substance and checking the existence of the biopolymer.

When an electrochemical change is detected by using the above biopolymer, NADH in an NAD+/NADH cycle can be used. That is, an electric change caused by oxidizing or reducing NADH produced by an enzyme bound to a biopolymer with an electrode itself may be measured. The substance which involves this electrochemical oxidation/reduction reaction is not limited to these.

The above change may be detected with a labeling agent used to label a nucleic acid probe itself without using the above intercalating agent. A substance capable of directly or indirectly detecting a signal is an electrode active substance such as ferrocene or viologen.

Hybridization can be detected by the response to the electrode of an intercalating agent bound to a double-stranded nucleic acid.

In the present invention in which electrodes are used, a measuring system which is composed of a potentiostat, function generator and recorder may be used.

The determination of the detected gene may be carried out by setting the potential to around the oxidation/reduction potential of an intercalating agent and measuring an oxidation/reduction current.

The constitution disclosed in Japanese Patent Laid-Open No. 05-285000 may be employed as specific example of the constitution in which the above intercalating agent is used and the gold-containing film according to the present invention is used as electrodes.

### EXAMPLES

Specific examples will be provided hereinafter for the purpose of further illustrating the present invention but are in no way to be taken as limiting. According to the present inventions, the substitution and design change of each element may be made within such a range as to attain the object of the present invention. Reference symbols used in the examples are the same as those in FIG. 1.

### Example 1

The apparatus shown in FIG. 1 was used first to form a metal single crystal thin film.

40 g of potassium iodide and 6 g of iodine were added into 500 ml of distilled water, stirred and dissolved. 3 g of gold was added to the resulting solution and dissolved by stirring. After dissolution, 100 ml of the solution was collected from this solution and fed to a reactor, and 500 ml of distilled water was further added to this solution and stirred to prepare a crystal growth solution 14 which was added into the solution tank 12.

An Si substrate 10 was used and immersed in this crystal growth solution 14. Thereafter, this solution was heated at 80° C. and left to stand. When the substrate was taken out after 1.5 hours and observed, (111)-oriented single crystals were formed on the Si substrate. A grain boundary was formed between the single crystals. It was found by STM observation that the surface unevenness of each single crystal was 0.4 nm/μm².

An oligomer which was a 75-mer of thymine (to be abbreviated as “T” hereinafter) having a thiol group bonded to a hydroxyl group at the 5′ terminal through a phosphate group and hexamethylene was prepared as a DNA probe. This single-stranded DNA was added to a solution containing 7.5% by weight of glycerin, 7.5% by weight of urea, 7.5% by weight of ethylene glycol and 1% by weight of acetylene alcohol (trade name: Acetylenol EH; manufactured by Kawasaki Fine Chemical Co., Ltd.) in a concentration of 8 μM and dissolved in the solution. The BC-50 printer head (manufactured by Canon Inc.) for the BJF-850 bubble jet printer (manufactured by Canon Inc.) using a bubble jet method which is a kind of thermal jet method was modified to discharge several hundreds of μl of a solution. This modified head was mounted to a discharge drawing machine that is so modified that the solution could be discharged to the above substrate. The above DNA solution was injected into a modified tank of this head in an amount of several hundreds of μl and spotted. The discharge amount of the solution at the time of spotting was 4 pl/droplet and the solution was spotted to a 10 mm×10 mm area of the center portion of the substrate. The diameter of the spotted dots was about 50 μm.

The formed DNA chip was observed through the scanning probe microscope manufactured by Digital Instruments Co., Ltd. using a tapping mode AFM technique. A silicon single crystal probe (trade name: D-NCH) was used in the chip. As a result, an image of DNA formed on the gold atomic step was obtained.

### Example 2

The apparatus shown in FIG. 2 was used to carry out the first step. The gate valve 24 was opened to introduce the Si substrate 10 into the latent image chamber 21 and mount it on the sample holder 20, and the gate valve 24 was closed. The latent image chamber 21 was evacuated by the vacuum evacuator 25 until the inside pressure of the latent image chamber 21 became 10⁻⁷ Torr or less. Oxygen was introduced into the latent image chamber 21 from the gas introduction port 22 at a flow rate of 800 sccm, and the evacuation rate of the vacuum evacuator 25 was controlled to set the inside pressure to 10 Torr. Then, a laser beam having a wavelength of 248 nm oscillated by the KrF excimer laser light source 26 was uniformly applied to the mask 28 having a desired pattern by the illumination optical system 27, a pattern image of the mask 28 was formed on the substrate 10 by the projection optical system 29, and the substrate 10 was irradiated with light for 10 minutes (the intensity of irradiation light on the surface of the Si substrate 10 was 100 mW/cm²) to form a latent image layer. After the irradiation, the supply of the gas was stopped and the latent image chamber 21 was evacuated until the inside pressure became 10⁻⁷ Torr or less. A nitrogen gas was introduced into the latent image chamber 21 to return its inside pressure to atmospheric pressure, and the gate valve 24 was opened to take out the Si substrate having the latent image layer formed thereon.

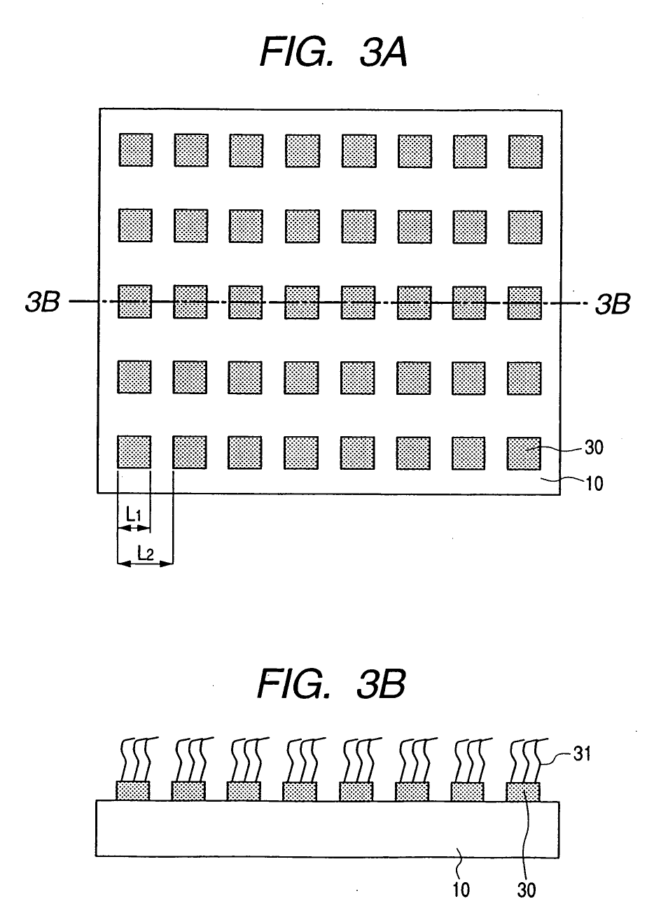

Thereafter, the apparatus shown in FIG. 1 was used to form a gold thin film on the Si substrate in the same manner as that of Example 1. FIG. 3A is a schematic plane view of the formed patterned substrate. The formed pattern had a length L1 of 100 μm and a length L2 of 500 μm.

An oligomer which was a 75-mer of thymine (to be abbreviated as “T” hereinafter) having a thiol group bonded to a hydroxyl group at the 5′ terminal through a phosphate group and hexamethylene was prepared as a DNA probe. By using this, a similar solution to that of Example 1 to be discharged by a bubble jet printer was prepared, and a liquid containing a probe nucleic acid was spotted to a glass plate by using a bubble jet printer so that a probe nucleic acid was formed on a gold thin film at intervals of the pattern shown in FIG. 3A by moving the substrate having the gold pattern formed in the previous step by a drive unit not shown two-dimensionally to thereby form a probe array on the substrate.

FIG. 3B is a schematic sectional view taken along the line 3B-3B of FIG. 3A of the finally formed probe array.

The formed DNA chip was observed through the scanning probe microscope manufactured by Digital Instruments Co., Ltd. using a tapping mode AFM technique. A silicon single crystal probe (trade name: D-NCH) was used in the chip. As a result, an image of DNA formed on the gold atomic step of the patterned gold was obtained.

### Example 3

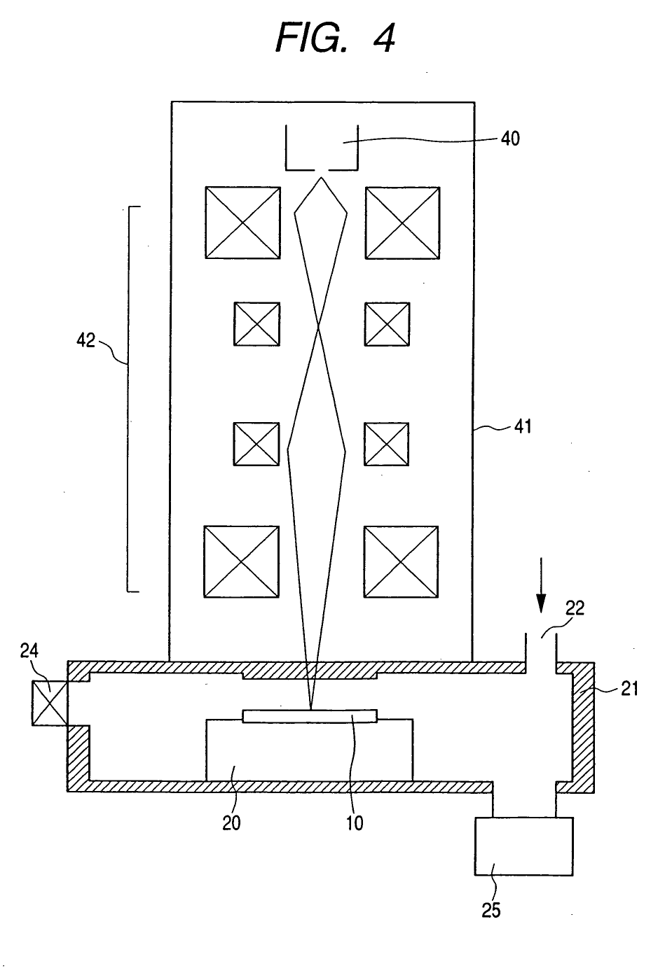

An example in which a patterning apparatus using an electron beam shown in FIG. 4 was used will be explained. The Si substrate 10 was mounted on the sample holder 20, and the gate valve 24 was closed. The latent image chamber 21 was evacuated by the vacuum evacuator 25 until the inside pressure became 10⁻⁷ Torr or less. Oxygen was introduced from the gas introduction port 22 at a flow rate of 10 sccm to set the inside pressure of the latent image chamber 21 to 3×10⁻³ Torr. Then, an electron beam generator 41 was activated to generate electrons with an electron gun 40, an electron beam 43 was accelerated to 2 kV by an electronic optical system 42 and converged to a spot diameter of 0.4 μm on the surface of a film, and a stage having the sample holder 20 mounted thereon was moved two-dimensionally by the drive unit in cooperation with an electron beam shutter (not shown) to form a latent image layer having a desired pattern without using a mask. After the irradiation, the supply of the gas was stopped, and the latent image chamber 21 was evacuated until the inside pressure became 10⁻⁷ Torr or less. A nitrogen gas was introduced into the latent image chamber 21 to return its inside pressure to atmospheric pressure and the gate valve 24 was opened to take out the Si substrate having the latent image layer formed thereon. The subsequent steps were carried out in the same manner as those of Example 2, and it was confirmed that a probe array was formed on the substrate.

### Example 4

A gold pattern formed film was formed on the Si substrate in the same manner as that of Example 3. The formed pattern had the length L1 of 50 μm and the length L2 of 130 μm in FIG. 3A.

A single-stranded DNA having the following sequence was synthesized by a DNA automatic synthesizer. A thiol (SH) group was introduced into the terminal of the single-stranded DNA having SEQ ID NO: 1 by using the Thiol-Modifier (manufactured by Glen Research Co., Ltd.) during synthesis by the DNA automatic synthesizer. Subsequently, ordinary deprotection was carried out to collect DNA which was purified by high pressure liquid chromatograph.

SEQ ID NO: 1
5′ HS—(CH₂)₆—O—PO₂—O-ACTGGCCGTCGTTTTACA3′

Subsequently, the DNA automatic synthesizer was used to synthesize single-stranded nucleic acids having SEQ ID NO: 2 to SEQ ID NO: 4. The single-stranded nucleic acid having SEQ ID NO: 2 was obtained by changing one base of the single-stranded nucleic acid having SEQ ID NO: 1, the single-stranded nucleic acid having SEQ ID NO: 3 was obtained by changing three bases of the single-stranded nucleic acid having SEQ ID NO: 1, and the single-stranded nucleic acid having SEQ ID NO: 4 was obtained by changing six bases of the single-stranded nucleic acid having SEQ ID NO: 1. The Thiol-Modifier (manufactured by Glen Research Co., Ltd.) was used to introduce a thiol (SH) group into the terminals of the single-stranded DNA's having SEQ ID NO: 1 to SEQ ID NO:4 during synthesis by the DNA automatic synthesizer. Subsequently, ordinary deprotection was carried out to collect DNA's which were purified by high pressure liquid chromatograph and used in the following experiments. SEQ ID NO: 2 to SEQ ID NO: 4 are shown below.

SEQ ID NO: 2
5′ HS—(CH₂)₆—O—PO₂—O-ACTGGCCGTTGTTTTACA3′
SEQ ID NO: 3
5′ HS—(CH₂)₆—O—PO₂—O-ACTGGCCGCTTTTTTACA3′
SEQ ID NO: 4
5′ HS—(CH₂)₆—O—PO₂—O-ACTGGCATCTTGTTTACA3′

Using the above single-stranded DNA's having SEQ ID NO: 1 to SEQ ID NO: 4, four different liquids to be discharged were prepared in the same manner as that of Example 1 above and charged into four ink tanks for a bubble jet printer, and the ink tanks were mounted in the respective bubble jet heads.

Thereafter, the substrate was moved two-dimensionally by the drive unit (not shown) so that the liquid could be spotted to the patterned substrate to spot four kinds of probes to the gold thin film in order to prepare a probe array.

A single-stranded DNA having a complementary base sequence to DNA having SEQ ID NO: 1 was synthesized by the DNA automatic synthesizer, and rhodamine was bound to the 5′ terminal to obtain a labeled single-stranded DNA. This labeled single-stranded DNA was dissolved in a 1M NaCl/50 mM phosphate buffer solution (pH 7.0) to a final concentration of 1 μM in order to carry out a hybridization reaction with the obtained probe array for 3 hours. Thereafter, the probe array was washed with the 1M NaCl/50 mM phosphate buffer solution (pH 7.0) to wash away a single-stranded DNA which was not hybridized with the probe nucleic acid. Each spot of the probe array was then observed through a fluorescence microscope (manufactured by Nikon Corporation) and the amount of fluorescence was determined by using an inverted fluorescent microscope having a filter set suitable for Rhodamine B while connecting it to an image analyzer (trade name: ARGUS 50; manufactured by Hamamatsu Photonics Co., Ltd.).

The amount of fluorescence on the spot of the DNA probe having SEQ ID NO: 1 which perfectly matched the labeled single-stranded DNA was 4,600, whereas the amount of fluorescence on the spot of the DNA probe having SEQ ID NO: 2 and one different base was 2,800. The amount of fluorescence on the spot of the DNA probe having sequence No. 3 and three different bases was 2,100 which was not more than half of that of the perfectly matching DNA probe, and fluorescence was not observed for DNA having SEQ ID NO: 4 and six different bases. It was confirmed from the above results that specifically a single-stranded DNA having a perfectly complementary base sequence could be detected on the DNA array substrate.

### Example 5

A plurality of gold thin film pattern electrodes were formed on the Si substrate in the same manner as that of Example 1 using the same apparatus shown in FIG. 1 as in Example 2.

Leads were formed with silver paste and connected to the respective thin film pattern electrodes.

The single-stranded DNA's having SEQ ID NO: 1 and SEQ ID NO: 4 were used as in Example 4 to prepare two different liquids to be discharged in the same manner as those of the above examples, the liquids were charged into two ink tanks for a bubble jet printer, and the ink tanks were set in the respective bubble jet heads. Thereafter, a substrate manufactured in the same manner as in Example 1 was set in the printer, and two different DNA probe solutions were spotted to gold thin film pattern electrodes at different positions on the substrate to produce a probe array.

A single-stranded DNA having a complementary base sequence to the base sequence of the DNA probe having SEQ ID NO: 1 was synthesized by the DNA automatic synthesizer and (6-aminohexyl)dATP was introduced into the 3′-terminal of the DNA using a terminal dioxynucleotidyl transferase to obtain a labeled single-stranded DNA having aminoacridine bonded to the amino group through glutaraldehyde.

This labeled single-stranded DNA was dissolved in the 1M NaCl/50 mM phosphate buffer solution (pH 7.0) to a final concentration of 1 μM in order to carry out a hybridization reaction with the obtained probe array for 3 hours. After the end of the reaction, an oxidization/reduction current owing to aminoacridine used to label the nucleic acid probe was measured.

As a result, in the electrodes to which the DNA probe having SEQ ID NO: 1 was bound, a specifically different current value could be detected.

According to the probe carrier, the method of producing the same, the method of evaluating the same and the method of detecting a target substance using the same of the present invention, there can be provided the probe carrier having the single-stranded DNA probe firmly bound to the carrier by reacting the single-stranded DNA probe having the thiol group as the functional group with the gold thin film capable of forming smooth surface to form the strong bond between them through sulfur atom. Since the gold thin film used in the present invention has extremely high flatness and is so stable that it is hardly oxidized in the air, the direct evaluation of the probe through the microscope having atomic resolution such as the scanning probe microscope is enabled.

The probe array having a plurality of stable probes can be produced by patterning gold.

1-19. (canceled)

20. A method of producing a nucleic acid probe array comprising the steps of:
forming a gold-containing film array by two-dimensionally arranging a gold-containing film; and
applying a nucleic acid probe having a thiol group to the gold-containing film by using an ink jet technique.

21. The method according to claim 20, wherein the nucleic acid probe is a single-stranded DNA probe, and the gold-containing film is a thin film containing (111)-orientated single crystal gold, and wherein the method comprises forming the gold-containing film on a carrier, and immobilizing the nucleic acid probe to the gold-containing film through a sulfur atom.

22. The method according to claim 20, wherein surface unevenness of the gold-containing film is 0.5 nm or less per μm².

23. The method according to claim 20, wherein the gold-containing film is formed on a carrier by immersing the carrier in a gold complex solution.

24. The method according to claim 20, wherein the gold containing-film is patterned by applying an electron beam or ion to a carrier on which the gold-containing film is formed.
